(19) United States
(12) Patent Application Publication
(10) Pub. No.: US 2005/0255150 A1
(43) Pub. Date: Nov. 17, 2005

(54) Wound treatment patch for alleviating pain

(76) Inventors: Douglas R. Cassel, Hummelstown, PA (US); Kirti H. Valia, Plainsboro, NJ (US)

Correspondence Address: Alexander R. Pagano, Esq.;Suite 230A, Bldg. A, 30 Vreeland Road, Florham Park, NJ, 07932, US

(21) Appl. No.: 11/113,933

(22) Filed: Apr. 25, 2005

Related U.S. Application Data
(60) Provisional application No. 60/565,323, filed on Apr. 26, 2004.

(51) Int. Cl. A61K 31/445; A61K 9/70

(52) U.S. Cl. 424/449; 514/317

(57) ABSTRACT

Methods and topical patches for outpatient treatment of moderate to severe pain with an opioid, for example, the pain caused by surgically closed wounds. The methods and patches of the invention comprise: (1) a local-anesthetic component for local delivery of a local anesthetic; and (2) an opioid component for transdermal delivery of an opioid. The synergistic combination of a local anesthetic and an opioid provides effective relief of moderate to severe pain at lower opioid doses than current opioid treatments.

This application claims the benefit of U.S. Provisional Application No. 60/565,323 filed Apr. 26, 2004, entitled Wound Treatment Patch For Alleviating Pain, by R. Douglas Cassel, which application is hereby incorporated herein by reference in its entirety.

### FIELD

The invention relates to a topical patch for transdermal delivery of opioid pain medications. The patches of the invention are useful for treating pain, particularly, post-operative pain.

### BACKGROUND

Today's managed-care principles apply cost pressures directed toward the most inexpensive level of health care. For example, managed care companies direct surgery patients to physicians' offices or freestanding surgery centers, then treatment on an outpatient basis. Surgery centers are geared toward surgeries performed during a one-day visit, without a hospital stay. Basically, insurers do not want to pay for an expensive hospital surgical suit.

Unfortunately, effective treatment of the pain resulting from surgical procedures is generally not possible on an outpatient basis because the most effective pain relievers, opioids, require doctor supervision in a hospital.

Pain results from the noxious stimulation of nerve endings. Nociceptive pain is caused by noxious stimulation of nociceptors (e.g., a needle stick or skin pinch), which then transmit impulses over intact neural pathways to the spinal neurons and then to the brain. Neuropathic pain is caused by damage to neural structures, such as damage to peripheral nerve endings or nociceptors, which become extremely sensitive to stimulation and can generate impulses in the absence of stimulation (e.g., herpes zoster pain after the rash has healed). Peripheral nerve damage can lead to pathological states where there is a reduction in pain threshold (i.e., allodynia), an increased response to noxious stimuli (hyperalgesia), or an increased response duration (persistent pain). GOODMAN & GILMAN'S THE PHARMACOLOGICAL BASIS OF THERAPEUTICS 529 (Joel G. Hardman et al. eds., 9th ed., 1996); HARRISON'S PRINCIPLES OF INTERNAL MEDICINE 53-58 (Anthony S. Fauci et al. eds., 14th ed. 1998).

Pain relief by topical drug administration offers distinct advantages over conventional administration methods. For example, intravenous and subcutaneous administration by injection is not only painful and invasive but requires administration by a medical professional and thus is not suitable for the outpatient treatment so desired by the managed-care companies. Furthermore, when treating localized conditions by oral and intravenous administration, the drug is circulated systemically rather than restricted to the painful area, leading to undesired side effects. TRANSDERMAL AND TOPICAL DRUG DELIVERY SYSTEMS (Tapash K. Ghosh et al. eds., 1997).

In general, drug administration via the skin is divided into two categories: 1) transdermal administration; and 2) intradermal administration. Transdermal administration involves transport through the skin and into the blood stream to treat conditions systemically. One the other hand, intradermal administration is intended to impart a cutaneous effect, while keeping the pharmocological effects of the drug localized to the intracutaneous regions of drug penetration and deposition. TRANSDERMAL AND TOPICAL DRUG DELIVERY SYSTEMS (Tapash K. Ghosh et al. eds., 1997).

It is universally accepted by physicians that opioids are among the drugs most effective for treating moderate to severe pain. For many patients, opioid analgesics are the most effective and often the only treatment that provides significant relief of moderate to severe pain. Richard C. Stephenson, Opioids in End of Life Care: Promises and Problems, 65 NC MED. 229-234, 231 (2004). Unfortunately, the currently available opioid treatments require doctor supervision in a hospital. This is because the opioid doses required for effective relief of moderate to severe pain are at a level where there is a risk of serious side effects, such as hypoventilation. This greatly conflicts with the managed-care principals discussed above. For example, because of the dose levels delivered by the Duragesic® fentanyl patch, it is contraindicated for outpatient use for fear of life threatening hypoventilation that might occur in the absence of doctor supervision in a hospital. See PHYSICIAN'S DESK REFERENCE 1573-1577 (Medical Economics, 59th ed. 2005).

What is needed is a transdermal opioid treatment that provides low opioid doses, such that it is suitable for outpatient use, yet is effective for treatment of moderate to severe pain resulting from post-operative wounds.

### SUMMARY

The invention relates to topical methods and topical patches for treatment of moderate to severe pain with an opioid, for example, the moderate to severe pain caused by surgically closed wounds.

The methods and patches of the invention comprise: (1) a local-anesthetic component for local delivery of a local anesthetic; and (2) an opioid component for transdermal delivery of an opioid. The synergistic combination of a local anesthetic and an opioid provides effective relief of moderate to severe pain at lower opioid doses than current opioid treatments. As such, the methods and patches of the invention are suitable for outpatient use.

Preferably, the local anesthetic is delivered directly to the pain site for local pain treatment. For example, for treatment of moderate to severe pain caused by a surgically closed wound, the local anesthetic component is preferably contacted directly with the wound. Preferably, the opioid is transdermally delivered through intact skin. Thus, preferably, the opioid component of a patch of the invention is contacted with intact skin.

In one embodiment, the invention relates to a patch comprising a local anesthetic and an opioid, wherein the opioid is present in an amount sufficient for transdermal delivery and systemic treatment of moderate to severe pain and the local anesthetic is present in an amount sufficient for intradermal delivery and local treatment of moderate to severe pain.

In another embodiment, the invention relates to a method of treating, ameliorating or alleviating moderate to severe pain in a mammal comprising locally administering a therapeutically effective amount of a local anesthetic to the site of the pain and transdermally administering a therapeutically effective amount of an opioid to intact skin by way of one or more patches to a mammal in need of such treatment.

In still another embodiment, the invention relates to the use of a local anesthetic and an opioid in the preparation of a medicament patch for a method of treating, ameliorating or alleviating moderate to severe pain in a mammal comprising locally administering a therapeutically effective amount of a local anesthetic to the site of the pain and transdermally administering a therapeutically effective amount of an opioid to intact skin by way of one or more patches to a mammal in need of such treatment.

In yet one more embodiment, the invention is directed to a kit comprising a local anesthetic component and an opioid component, wherein the opioid component comprises an opioid for transdermal delivery and the local anesthetic component comprises a local anesthetic for local delivery, and wherein the amounts of the opioid and the local anesthetic are sufficient for treatment of moderate to severe pain caused by a surgically closed wound.

### BRIEF DESCRIPTION OF THE FIGURES

These and other features, aspects, and advantages of the present invention will become better understood with regard to the following description, appended claims, and accompanying drawings where:

FIG. 1 depicts the application surface of one embodiment of a patch of the invention;

FIG. 2 is a transectional view of one embodiment of a patch of the invention;

FIG. 3 depicts the application surface of a further embodiment of a patch of the invention; and

FIG. 4 is a transactional view of a further embodiment of a patch of the invention.

### DETAILED DESCRIPTION

1. Definitions

1.1 Intradermal Administration

As used herein, the term “intradermal administration” means topical administration of a pharmaceutical to a mammal, to deliver the pharmaceutical to the local tissue under and around the site of administration. Preferably, intradermal administration is effected without absorption of the pharmaceutical into the mammal's blood stream. The purpose of intradermal administration is to elicit a local affect in contrast to transdermal administration where the objective is to transfer the pharmaceutical through the skin and into the blood stream for a systemic effect.

1.2 Local Anesthetic

As used herein, the term “local anesthetic” means any drug that provides local numbness or analgesia or any drug that provides a regional blockage of nociceptive pathways (afferent and/or efferent). The local anesthetic can be any local anesthetic known or to be developed. Examples of local anesthetics suitable for use with the invention include: ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocalne, procaine, propanocaine, proparacaine, propipocaine, propoxycaine, pseudococaine, pyrrocaine, ropivacaine, salicyl alcohol, tetracaine, tolycaine, trimecaine, zolamine, or a pharmaceutically acceptable salt thereof, or a mixture thereof.

The amide and ester type local anesthetics are preferred. Amide type local anesthetics are characterized by an amide functionality, while ester type local anesthetics contain an ester functionality. Preferred amide type local anesthetics are: lidocaine, bupivacaine, prilocalne, mepivacaine, etidocaine, ropivacaine, dibucaine, and mixtures thereof. Preferred ester type local anesthetics are: tetracaine, procaine, benzocaine, chloroprocaine, their pharmaceutically acceptable salt, or a mixture thereof. The most preferred local anesthetic is lidocaine.

The meaning of “local anesthetic” also encompasses drugs not traditionally associated with local anesthetic properties but which have a local anesthetic effect, for example, non-narcotic analgesics, such as, acetylsalicylic acid, ketoprofen, piroxicam, diclofenac, indomethacin, ketorolac, rofecoxib, and celecoxib, and pharmaceutically acceptable salts thereof, or mixtures thereof.

1.3 Mammal

As used herein, the term mammal means any mammal, for example, but not limited to humans; pets, such as dogs and cats; farm mammals, such as horses, cows, pigs, and sheep; and laboratory animals, such as monkeys, guinea pigs, rats, and mice. Preferably, a “mammal” is a human.

1.4 Opioid

As used herein the term “opioid” means all agonists and antagonists of opioid receptors, such as mu (μ), kappa (κ), and delta (δ) opioid receptors and subtypes thereof. For a discussion of opioid receptors and subtypes see Goodman and Gilman's The Pharmacological Basis of Therapeutics 9th ed. J. G. Harman and L. E. Limird Eds., McGraw-Hill New York pp. 521-555 (1996), hereby incorporated herein by reference.

The opioid can be any opioid receptor agonist or antagonist known or to be developed. Preferred opioids interact with the μ-opioid receptor, the κ-opioid receptor, the delta (δ) opioid receptors or all three. Preferably, the opioid is an opioid-receptor agonist.

Examples of suitable opioids for use with the invention include, but are not limited to, fentanyl, alfentanil, allylprodine, alphaprodine, anileridine, benzylmorphine, benzitramide, norbinaltorphimine, bremazocine, buprenorphine, butorphanol, clonitazene, codeine, CTOP, DAMGO, desomorphine, dextromoramide, dezocine, diampromide, dihydrocodeine, dihydrocodeine enol acetate, dihydromorphine, dimenoxadol, dimepheptanol, dimethylthiambutene, dioxaphetyl butyrate, dipipanone, diprenorphine, DPDPE, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levorphanol, lofentanil, loperamide, meperidine, meptazinol, metazocaine, methadone, metopon, morphine, myrophine, nalbuphine, naltrindole, benzoylhydrazone, naltrexone, narceine, nicomorphine, norlevorphanol, normethadone, normorphine, norpipanone, opium, oxycodone, oxymorphone, papaveretum, papaverine, pentazocine, phenadoxone, phenazocine, phenoperidine, piminodine, pirtramide, proheptazine, promedol, propiram, propoxyphene, remifentanil, spiradoline, sufentanil, tilidine, amiphenazole, cyclazocine, levallorphan, nalmefene, nalorphine, naloxone, loperamide, and naltrexone and pharmaceutically acceptable salts thereof, and mixtures thereof.

Preferred opioids include fentanyl, buprenorphine, sufentanil, hydromorphone, and pharmaceutically-acceptable salts thereof, and mixtures thereof. The most preferred opioid is fentanyl and pharmaceutically-acceptable salt thereof.

1.5 Patch

As used herein “patch” comprises at least a topical drug formulation and a covering layer, such that, the patch can be placed over the application site, such as over a surgically closed wound, thereby positioning the patch/drug formulation adjacent to the wound's surface.

1.6 Pharmaceutically Acceptable Salt(s)

The phrase “pharmaceutically acceptable salt(s)”, as used herein, unless otherwise indicated, means those salts that retain the biological effectiveness and properties of neutral local anesthetics and that are not otherwise unacceptable for pharmaceutical use. Pharmaceutically acceptable salts include salts of acidic or basic groups, which groups may be present in the local anesthetics. The local anesthetics used in present invention that are basic in nature are capable of forming a wide variety of salts with various inorganic and organic acids. Pharmaceutically acceptable acid addition salts of basic local anesthetics and basic opioids used in the present invention are those that form non-toxic acid addition salts, i.e., salts comprising pharmacologically acceptable anions, such as the hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. The local anesthetics and opioids of the present invention that include an amino moiety may form pharmaceutically acceptable salts with various amino acids, in addition to the acids mentioned above. Suitable base salts are formed from bases which form non-toxic salts and examples are the aluminum, calcium, lithium, magnesium, potassium, sodium, zinc and diethanolamine salts. For a review on pharmaceutically acceptable salts see Berge et al., 66 J. PHARM. SCI. 1-19 (1977), hereby incorporated herein by reference.

1.7 Surgically Closed Wound

The term “surgically closed wound”, as used herein, means any wound that has been closed, such that, the opposing edges of skin—which comprise the wound—have been joined together by a device or material. Preferably, the wound has been closed by a medical professional, such as, a surgeon, doctor, doctors assistant, emergency medical technician, or nurse. Surgically closed wounds include but are not limited to wounds that have been closed with: sutures (absorbable or non-absorbable, synthetic or natural), staples, and biological glues. Examples of wounds requiring surgical closure include, but are not limited to, wounds resulting from laparoscopy, hemiaplasty, breast biopsy, and excision of a subcutaneous tumor

1.8 Therapeutically Effective Amount with Respect to a Local Anesthetic

As used herein, a “therapeutically effective amount” with respect to a local anesthetic means the amount required in a patch of the invention for local delivery to the pain site that, when used in combination with an opioid according to the invention, is sufficient to treat or ameliorate pain in a mammal.

1.9 Therapeutically Effective Amount with Respect to an Opioid

As used herein, a “therapeutically effective amount” with respect to an opioid means the amount required in a patch of the invention for transdermal delivery that, when used in combination with a local anesthetic according to the invention, is sufficient to treat or ameliorate pain in a mammal.

1.10 Topically Acceptable

As used herein, the phrase “topically-acceptable” means any pharmaceutical, excipient or other component of a topical formulation that is safe or approved for intradermal or transdermal administration in mammals.

1.11 “Topical Administration” or “Topical Delivery”

As used herein, the term “topical administration” or “topical delivery” means intradermal or transdermal administration of a pharmaceutical by administration of the pharmaceutical or a composition comprising the pharmaceutical.

1.12 Topical Composition

The term “topical composition” means a pharmaceutical composition designed for intradermal or transdermal administration of a drug or pharmaceutical in a mammal, which contains the pharmaceutical.

1.13 Transdermal Administration

As used herein, the term “transdermal administration” means topical administration of a pharmaceutical to a mammal, to systemically deliver the pharmaceutical into the mammal's blood stream. The purpose of transdermal administration is to elicit a systemic affect in contrast to intradermal administration where the objective is to elicit a local effect.

2. Patches of the Invention

Patches of the invention preferably comprise: (1) a backing layer, having an adhesive thereon; (2) a local-anesthetic component for local delivery of a local anesthetic, preferably, a local anesthetic in a carrier, referred to herein as a “local anesthetic composition”; (3) an opioid component for systemic delivery of an opioid, preferably, an opioid in a carrier, referred to herein as an “opioid composition”; and (4) a drug permeable membrane overlaying the active components. The opioid component and the local-anesthetic component are collectively referred to herein as the “active components”. The opioid component and the local-anesthetic component can be within the same patch (“two-component patch”) or in different patches. These components are described in more detail below.

Patches of the invention can be any shape or size or customized to fit irregularly shaped wounds. For example, patches of the invention can be rectangular in shape to treat the pain caused by longitudinal or vertical incisions resulting from ventral hernia surgery or an abdominal hysterectomy, or patches of the invention can circular in shape to treat the pain caused by laparoscopy, percutaneous access, angiography, or angioplasty.

Patches suitable for use in the invention include, but are not limited to, (1) the matrix type patch; (2) the reservoir type patch; (3) the multi-laminate drug-in-adhesive type patch; and (4) the monolithic drug-in-adhesive type patch; TRANSDEPMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

The matrix patch comprises a drug containing matrix, an adhesive backing film overlay, and preferably, a release liner. In some cases, it may be necessary to include a impermeable layer to minimize drug migration into the backing film (e.g., U.S. Pat. No. 4,336,243, incorporated herein by reference). The drug-containing matrix is held against the skin by the adhesive overlay. Examples of suitable matrix materials include but are not limited to lipophilic polymers, such as polyvinyl chloride, polydimethylsiloxane, and hydrophilic polymers like polyvinylpyrrolidone, polyvinyl alcohol, hydrogels based on gelatin, or polyvinylpyrrolidone/polyethylene oxide mixtures.

The reservoir type patch design is characterized by a backing film coated with an adhesive, and a reservoir compartment comprising a drug formulation preferably, in the form of a solution or suspension, that is separated from the skin by a semipermeable membrane (e.g., U.S. Pat. No. 4,615,699, hereby incorporated herein by reference). The adhesive coated backing layer extends around the reservoir's boundaries to provide a concentric seal with the skin and hold the reservoir adjacent to the skin.

The monolithic drug-in-adhesive patch design is characterized by the inclusion of the drug formulation in the skin contacting adhesive layer, a backing film and preferably, a release liner. The adhesive functions both to release the anesthetic and adhere the anesthetic matrix to the skin. The drug-in-adhesive system does not require an adhesive overlay and thus the patch size is minimized. Also, drug-in-adhesive type patches are thin and comfortable (e.g., U.S. Pat. No. 4,751,087, incorporated herein by reference).

The multi-laminate drug-in-adhesive patch design further incorporates an additional semi-permeable membrane between two distinct drug-in-adhesive layers or multiple drug-in-adhesive layers under a single backing film. See Peterson, T. A. and Dreyer, S. J. 21 PROCEED. INTERN. SYMP. CONTROL. REL. BIOACT. MATER. 477-478 (Nice, France 1994), hereby incorporated herein by reference).

2.1 The Backing Layer

The backing layer or backing serves as the upper surface of the patch and functions as the primary structural element and provides the patch with its flexibility. The material selected for the backing material should be selected so that it is substantially impermeable to the local anesthetic and opioid and any other materials present, the backing is preferably made of a sheet or film of a flexible elastomeric material. The backing supports the active layers by way of an adhesive and holds the active layers against the application site. The combination of backing and adhesive should be biocompatible, non-irritating to the skin, breathable and able to hold the patch firmly against the skin.

Backings for use in patches of the invention are preferably flexible, biocompatible material that imitates the elastic properties of skin and conforms to the skin during movement. Preferred have a moisture-vapor transmission rate similar to human skin. This reduces the chance of an infection developing under the patch after it is applied to a patient's skin.

Preferably, the backing layer is derived from synthetic polymers like polyolefin oils polyester, polyethylene, polyvinylidine chloride, and polyurethane or from natural materials like cotton, wool, etc. Non-occlusive backings allow the area to breath (i.e., promote water vapor transmission from the skin surface). In one preferred embodiment, the backing film is an occlusive polyolefin foil (Alevo, Dreieich, Germany). The polyolefin foil is preferably about 0.6 to about 1 mm thick.

Other suitable backings are commercially available, for example, suitable backings can be purchased from 3M (St. Paul, Minn.) and Bertek (St. Albans, Vt.).

2.2 Permeable Membranes

Permeable membranes are used with patches of the invention to overlay the portion of the patch adjacent to the skin to permit delivery of the patch's active ingredients to the application site. Preferably, the permeable membrane comprises a breathable material that is agreeable to the surface of a surgically closed wound and permits local delivery of local anesthetic into the skin of the patient at the wound site and systemic delivery of opioid. Permeable membranes permit controlled delivery of the active components of the patch.

Permeable membranes, useful in the invention, include thin non-porous ethylene vinyl acetate films or thin micro-porous films of polyethylene and polyproylene. Preferably, the permeable membrane is an ethyl vinyl acetate copolymer membrane.

Suitable permeable membranes are commercially available, for example, suitable permeable membranes can be purchased from 3M (St. Paul, Minn.).

2.3 Adhesives

Adhesives are used with patches of the invention to adhere the active components to the backing and to adhere the backing to the patient's application site. Preferably, adhesives useful in the present invention can function under a wide range of conditions, such as, high and low humidity, bathing, sweating etc.

Adhesives for use with patches of the invention are well known in the art and selection is readily accomplished by an ordinary practitioner. Suitable adhesives include, but are not limited to, polyisobutylene-based adhesives, silicone-based adhesives, and acrylic-based adhesives.

Preferably the adhesive is a composition based on natural or synthetic rubber; a polyacrylate such as, polybutylacrylate, polymethylacrylate, poly-2-ethylhexyl acrylate; polyvinylacetate; polydimethylsiloxane; or and hydrogels (e.g., high molecular weight polyvinylpyrrolidone and oligomeric polyethylene oxide).

In one embodiment, pressure sensitive adhesives are preferred. Pressure sensitive adhesives are materials that adhere to a substrate by application of a light force and leave no residue when removed. Examples of pressure sensitive adhesives include, but are not limited to, Durotak® adhesives (e.g., Durotak® 2052, National Starch and Chemicals, Bridgewater, N.J.).

2.4 Carriers for the Active Components

In one embodiment of the invention, the opioid component and/or the local-anesthetic component comprises a pharmaceutically acceptable carrier to contain and deliver the active component to the application site.

Preferably, carriers are sterile and pharmaceutically acceptable for topical application and delivery of a drug into or through a patient's skin. Preferred functional characteristics of carriers are low adhesive strength, breathability, and conformability to the application area.

Pharmaceutically acceptable carriers for use in the invention are standard in the art, for example, matrix-type carriers, reservoir-type carriers, multi-laminate-type carriers, and monolithic drug-in-adhesive type carriers, such as those disclosed in TRANSDERMAL AND TOPICAL DRUG DELIVERY SYSTEMS (Tapash K. Ghosh et al. eds., 1997); see also Kristine Knutson and Lynn K. Pershing, Topical Drugs, in 2 REMINGTON: THE SCIENCE AND PRACTICE OF PHARMACY 866-885 (Alfonso R. Gennaro ed., 1995), the disclosures of which is hereby incorporated herein by reference.

In a preferred embodiment, the carrier is a matrix-type drug carrier. Matrix-type drug carriers are well known in the art. Suitable matrix-type drug carriers include, but are not limited to, the adhesives discussed above, such as polyisobutylene-based adhesives, silicone-based adhesives, and acrylic-based adhesives.

In another embodiment, the carrier is a hydrogel. Hydrogels are a mixture of water and a gelling agent, such as a hydrophilic polymer. In general, hydrogels form a three-dimensional lattice of polymer chains that retains an aqueous solution in a flexible, stable shape. Preferred hydrogels contain gelling agents distributed substantially uniformly throughout the carrier liquid, which is typically aqueous and my contain an alcohol and/or an oil.

Preferred gelling agents include, but are not limited to, crosslinked acrylic acid polymers such as carboxypolyalkylenes; hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Suitable hydrogels are commercially available, for example, suitable hydrogels can be purchased from BASF (St. Paul, Minn.) or Noveon (Cleveland, Ohio).

3. Dosage and Delivery Rates

The appropriate dosages of local anesthetic and opioid for pain treatment by way of patches of the invention is determined by a variety of factors. The rate at which the active components are absorbed is a function of skin permeability. Skin permeability varies between different sites on a patient's body and depends on the thickness of the stratum corneum. The stratum corneum is the outer-most layer of skin and is the main source of penetration and permeation resistance for dermally administered drugs. For example, the permeability, in general, increases in order from planter foot arch, lateral ankle, palm, ventral forearm, dorsal forearm, back, chest, thigh, abdomen, scalp, axilla, forehead, and scrotum; see R. C. Wester. & H. I. Maibach, Regional variation in Percutaneous Absorption, in PERCUTANEOUS ABSORPTION, MECHANISM, METHODOLOGY, DRUG DELIVERY 111-119 (R. L. Bronaugh & H. I. Maibach eds., 2nd ed. 1989), hereby expressly incorporated herein by reference.

For treatment of surgically closed wounds, dose size and frequency of dosages should be determined by a trained medical professional and will depend on many factors, including patient weight, wound location, wound size, wound severity and the type of surgical closure.

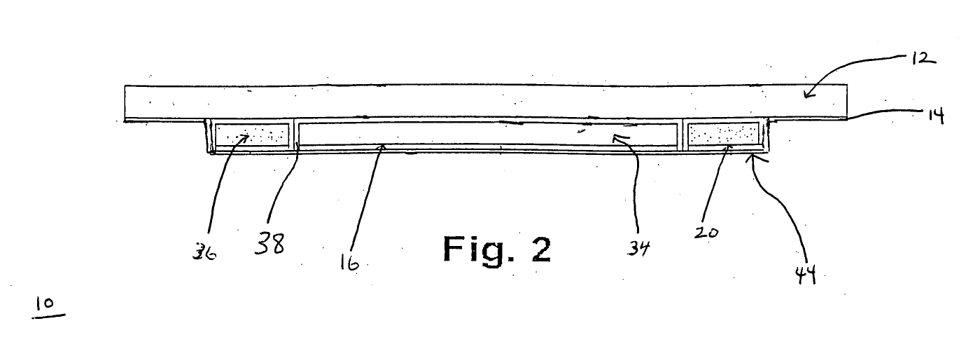

The delivery rate of local anesthetic and the opioid, from a patch of the invention, required for proper pain relief is determined by a variety of factors. With reference to FIG. 2, one important factor regarding delivery rate is the surface areas of the opioid and the local-anesthetic components 16 and 20 in contact with a patient's skin. In general, the larger the contact surface area, the higher the rate of delivery. Different delivery rates of local anesthetic and opioid may be needed depending on the severity of pain caused by the wound. The surface areas of components 16 and 20 can adjusted to provide the desired delivery rate of local anesthetic and systemic opioid to a patient.

3.1 Dosage and Delivery Rates of Local Anesthetic

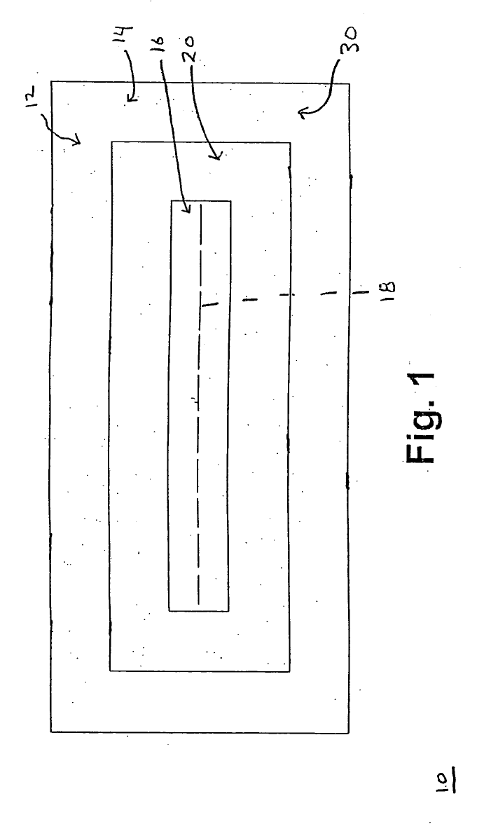

The dosage of local anesthetic administered by way of patches of the invention can be controlled by the active surface area of the local-anesthetic component of the patch (see 16 in FIG. 1) in contact with the skin. It is advantageous that several dosage strengths be available to the physician for his prescription, depending upon the severity of the pain. Thus, in general, a physician can adjust the local anesthetic dosage up or down by prescribing a patch having a local-anesthetic component of larger or smaller surface area.

In general, the local anesthetic component of patches of the invention will comprise local anesthetic in an amount of from about 0.1 mg/cm² to about 50 mg/cm².

For hydrogel-type patches of the invention, the local anesthetic component of patches of the invention will comprise local anesthetic in an amount of from about 0.5 mg/cm² to about 10 mg/cm², preferably, of from about 2 mg/cm² to about 8 mg/cm², more preferably, of from about 4 mg/cm² to about 6 mg/cm².

For matrix (drug-in-adhesive) type patches, preferably, the local anesthetic component of patches of the invention will comprise local anesthetic in an amount of from about 0.5 mg/cm² to about 30 mg/cm², preferably, of from about 5 mg/cm² to about 25 mg/cm², more preferably, of from about 8 mg/cm² to about 12 mg/cm².

3.2 Dosage and Delivery Rates of Opioid

The amount of opioid released from the patch is proportional to the surface area of the opioid component (see 20 in FIG. 1). The opioid release rate from a particular patch can be determined by methods well known in the art, such as those disclosed in U.S. Pat. No. 4,588,580 (May 13, 1986), hereby incorporated herein by references.

Opioid doses must be individualized by a medical professional based upon the status of each patient and should be assessed at regular intervals after patch application. Reduced opioid doses are suggested for the elderly.

In selecting an initial opioid dose, consideration should be given to: (1) the daily dosage, potency and characteristics of the opioid the patient has been taking previously, if any (e.g., whether it is a pure agonist or mixed agonist-antagonist); (2) the degree of opioid tolerance, if any; and (3) the general condition and medical status of the patient. Each patient should be maintained at the lowest dose providing acceptable pain control.

The typical initial dosage of the particular opioid for use in a patch of the invention is the amount that provides pain relief equivalent to that provided by about 10 mg to about 90 mg of morphine orally administered over a 24 hour period, preferably, about 20 mg to about 80 mg, more preferably, about 30 mg to about 50 mg orally administered over a 24 hour period.

Table 1 provides conversion factors for converting typical opioids into their respective morphine equivalents (with respect to the method of administration, whether oral or intramuscular) and the equivalent dose of fentanyl in patches of the invention.

TABLE 1
Conversion of Opioid to the Morphine Equivalent and
to the Fentanyl Equivalent

| | Intramuscular Administration | |
|---|---|---|
| | equivalent of 10 mg of | Oral Administration |
| | intramuscular | equivalent of 10 mg |
| Opioid | morphine in 24 hours | morphine in 24 hours |
| Morphine | 10 mg | 60 (30 mg) |
| Hydromorphone | 1.5 mg | 7.5 mg |
| Methadone | 10 mg | 20 mg |
| Oxycodone | 15 mg | 30 mg |
| Oxymorphone | 1 mg | 10 mg |
| Heroin | 5 mg | 60 mg |
| Meperidine | 75 mg | — |
| Codeine | 130 mg | 200 mg |
| The conversion ratio of 10 mg parenteral morphine equals 30 mg of oral morphine is based on clinical experience in patients with chronic pain. Physicians' Desk Reference 1573-1577 (59^{th} ed., Medical Economics, 2005). The conversion ratio of 10 mg parenteral morphine equals 60 mg of oral morphine is pasted on a potency study in acute pain. See Ashburn and Lipman, Management of Pain in the Cancer Patient, 76 ANESTH. ANALG. 402-416 (1993), hereby incorporated | | |
| # herein by reference. | | |

The proper release rate and dosage of opioid is determined based on a number of factors well known to those of skill in the art, including the identity of the opioid, its potency, and its rate of diffusion through the skin and into the circulatory system.

Preferably, the release rate of opioid from patches of the invention for treatment of the pain caused by surgically closed wounds is of from about 0.5 μg/h to about 3000 μg/h for every cm² of the opioid component in contact with the application site, more preferably, of from about 5 μg/h to about 500 μg/h, still more preferably of from about 10 μg/h to about 300 μg/h. Thus, dosage is controlled directly by adjustment of the surface area of the opioid component.

3.2.1 Dosage and Delivery Rates of Fentanyl

For use of fentanyl in the treatment of the pain induced by a surgically closed wound using a patch of the invention, the conversion factor is that 2 μg/cm²/hour of fentanyl administered via a patch of the invention is predicted to provide approximately equivalent pain relief to about 10 mg/24 hour dose of oral morphine equivalent.

Preferably, for the treatment of the pain caused by surgically closed wounds, the release rate of fentanyl from patches of the invention is 2.5 μg/h for every cm² of the opioid component in contact with the application site. Thus, dosage is controlled directly by adjustment of the surface area of the opioid component.

In the treatment of surgically closed wounds, the preferred initial dosage of fentanyl in patches of the invention is of from about 1 μg/hour to about 200 μg/hour over a 72 hour period, more preferably of from about 5 μg/hour to about 50 μg/hour, still more preferably, of from about 10 μg/hour to about 20 μg/hour over a 72 hour period. In general, the patches of the invention may be administered every 48 to 72 hours.

The equivalent doses of other opioids for transdermal administration by way of patches of the invention can readily be determined by methods well known in the art. See e.g., PHYSICIAN'S DESK REFERENCE 1573-1577 (Medical Economics, 59th ed. 2005); REMINGTON: THE SCIENCE AND PRACTICE OF PHARMACY (Alfonso R. Gennaro ed., 1995), both of which references are hereby incorporated herein by reference.

It should be noted that significant amount of opioid may be absorbed from the skin into the circulatory system for up to 20 hours after the patch has been removed, depending on the identity of the opioid, dosage and delivery rate.

4. Figures Depicting Patches of the Invention

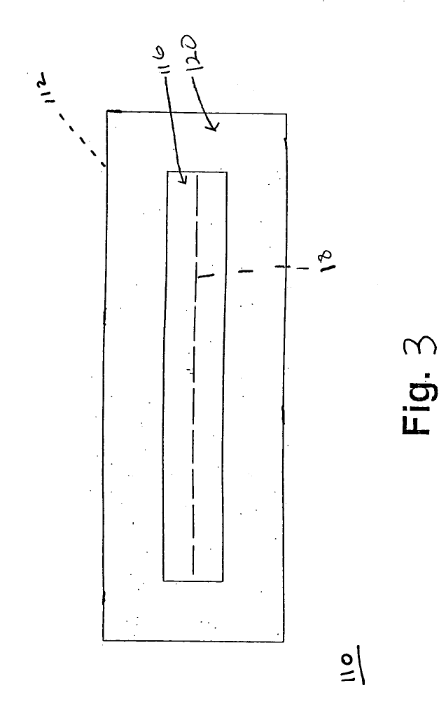

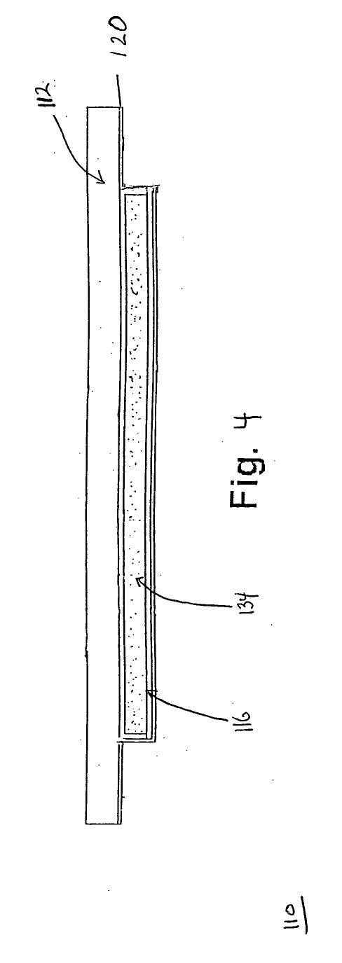

FIGS. 1-4 exemplify a few of the many embodiments of patches of the invention. Patches 10 (FIGS. 1-2) and 110 (FIGS. 3-4), depicted in the Figs., are useful to prevent or ameliorate pain, for example, incisional pain, such as the pain caused by surgically closed wound 18. Patches 10 and 110 comprises a local-anesthetic component 16 and an opioid component 20. When patches 10 and 110 are adhered to the application area, local anesthetic is delivered directly to longitudinal wound 18 to local pain receptors to alleviate pain locally. On the other hand, the opioid component 20 transdermally delivers opioid into the circulatory system, by way of the intact skin surrounding wound 18, for systemic pain alleviation. Because of the combined effects of the opioid acting systemically and local anesthetic acting locally, a lower systemic opioid dose is required to treat the pain as compared to traditional transdermal or oral delivery of opioid alone.

FIG. 1 is a depiction of the application surface of a patch 10. Patch 10 includes an elongated rectangular polyester film backing 12 having a polyvinylpyrrolidone-based adhesive surface 14.

The adhesive surface 14 of backing 12 includes an elongated local-anesthetic component 16, comprising a local anesthetic, preferably a local anesthetic in a local-anesthetic carrier, preferably a hydrogel or a matrix carrier. Local-anesthetic component 16 is centrally located on backing 12 and extends along the length of backing 12, such that it directly covers longitudinal wound 18.

Local-anesthetic component 16 is surrounded by opioid component 20. Opioid component 20 comprises an opioid, preferably, in an opioid carrier, preferably, a hydrogel or a matrix carrier. The local-anesthetic component 16 and the opioid component 20 are adhered to the adhesive surface 14 of backing 12 by way of an adhesive. The opioid component 20 has a smaller surface area than backing 12. As such, opioid component 20 is surrounded by rectangular adhesive area 30 of backing 12. Patch 10 is applied to the wound site such that area 30 is adhered to the skin of a patient to secure patch 10 in place over wound 18, whereby local anesthetic component 16 contacts wound 18, and opioid component 20 contacts intact skin.

FIG. 2 depicts a transectional view of a patch 10 of the invention, which comprises an elongated local-anesthetic component 16, which is adhered to backing 12 by way of adhesive 14. Local-anesthetic component 16 comprises local anesthetic reservoir 34, which comprises a measured dose of a local anesthetic. Patch 10 further comprises opioid component 20, which is also adhered to backing 12 by way of an adhesive and surrounds local-anesthetic component 16. Opioid component 20 comprises opioid reservoir 36 Opioid reservoir 36 comprises a measured dose of opioid.

The local anesthetic can be in concentrated form, but is preferably contained in local-anesthetic carrier. The combination of carrier and local anesthetic is herein referred to as the local-anesthetic composition. Similarly, the opioid can be in concentrated form, but is preferably contained in an opioid carrier. The combination of carrier and opioid is herein referred to as the opioid composition.

In one preferred embodiment, the local-anesthetic carrier is a matrix-type drug carrier, more preferably, a hydrogel.

In another preferred embodiment, local-anesthetic component 16 is shaped to correspond to the shape of a surgically closed wound 18. This allows a precise application of the local anesthetic along wound 18 to maximize the ability of the patch to deliver local anesthetic to pain receptors in wound 18.

Local anesthetic reservoir 34 and opioid reservoir 36 are defined within barrier 38. Barrier 38 comprises the same materials as are used for backing 14 and can be functionally designed to prevents migration of local anesthetic and opioid from one reservoir to the other by well-known methods, such as those described in U.S. Pat. No. 4,559,222 (issued Dec. 17, 1985), hereby incorporated herein by reference.

Permeable membrane 44 overlays local-anesthetic component 16 and opioid component 20. In use, permeable membrane 44 is positioned adjacent to wound 18 site, in contact with the skin and permits permeation of the local anesthetic and opioid to the application site at a selected delivery rate.

In another embodiment, opioid component 20 can be any kind of topical patch or component that controllably releases an opioid for systemic delivery, such as the Transtec® buprenorphine matrix-type pain patch manufactured by Gruenenthal GmbH.

Preferably, a release liner (not shown) overlays the entire bottom portion of the patch, including permeable membrane 44 and adhesive 14. The release liner is removed directly before topical application of the patch to the patient. Release liners prevent release of the active components during patch storage and maintain sterility after the patch is removed from its package. Preferably, the release liner is comprised of a sterile, impermeable flexible material that is easily removed. Suitable release liners are readily commercially available. Suitable release liners include, but are not limited to, occlusive, opaque, or clear polyester films with a thin coating of pressure sensitive release liner (e.g., silicone-fluorsilicone, and perfluorcarbon based polymers).

Preferably, patch 10 is sterilized and packaged in a protective package or sheath (not shown). The package ensures patch sterility until opened.

Reservoir 34 releases a sustained dosage of an local anesthetic into wound 18. Local anesthetic passes from local anesthetic composition 38 though permeable membrane 44 and into wound 18.

Reservoir 36 systemically delivers a sustained dosage of an opioid over time for systemic treatment of pain sensed at wound 18. Opioid passes through permeable membrane 44 then through the patient's skin for systemic delivery.

The specific position of local-anesthetic component 16 and opioid component 20 relative to each other on patch 10 is unimportant to the functionality of patch 10.

In a preferred embodiment, local-anesthetic component 16 and opioid component 20, comprising reservoirs 34 and 36 respectively, are mounted within the same patch a “two-component patch”.

In another embodiment, the opioid component 20 and the local-anesthetic component 16 are located in separate patches. The separate patches can be applied to two different areas of a patient's body. This is advantageous when surgically closed wound 18 is of a non-conventional shape such that the local-anesthetic component 16 must be custom made to conform to wound 18. Separate patches are also advantageous where the permeability of the skin surrounding wound 18 is not optimal for systemic administration of the opioid.

In sum, patch 10 dispenses a local anesthetic and a systemic opioid to the patient from first and second drug reservoirs 34 and 36. Referring to FIG. 1, in some applications, the local anesthetic can simply be contained in a paste or cream type topical drug formulation as disclosed in U.S. Pat. Nos. 6,383,511 and 6,645,521, which patents are hereby incorporated herein by references, Such a paste or cream can be spread on an adhesive backing 12 in area 16. The local anesthetic will flow directly through the skin and into the patient's circulatory system.

In an alternate embodiment, opioid component 20 is comprised of a paste or cream type opioid formulation for systemic administration of the opioid.

FIG. 3 is a depiction of the application surface of patch 110. Patch 110 includes an elongated rectangular polyester film backing 112 comprising centrally located, elongated local-anesthetic component 116. Local anesthetic component 116 comprises a local anesthetic, preferably a local anesthetic in a matrix carrier.

Local-anesthetic component 116 is surrounded by opioid component 120 comprising an opioid in a matrix adhesive. Patch 110 is applied to the wound site such that opioid component 120 adheres to the skin of a patient to secure patch 110 in place over wound 18, whereby local anesthetic component 116 contacts wound 18, and opioid component 120 contacts intact skin.

FIG. 4 depicts a transactional view of a patch 110, which comprises an elongated local-anesthetic component 116, which is adhered to backing 112. Local-anesthetic component 116 comprises local anesthetic reservoir 134, which comprises a measured dose of a local anesthetic. Patch 110 further comprises opioid component 120 comprising an opioid in a matrix adhesive.

Preferably, a release liner (not shown) overlays the entire bottom portion of the patch. The release liner is removed directly before topical application of the patch to the patient. Preferably, patch 110 is sterilized and packaged in a protective package or sheath (not shown). The package ensures patch sterility until opened.

Patches 10 and 110 are useful to treat incisional pain, such as the pain associated with surgically closed wounds. For example, patches 10 and 110 can be topically applied to a patient suffering pain in connection with surgically closed wound 18, by way of adhesive surfaces 14 or 120. The patch is secured such that local-anesthetic components 16 or 116 and opioid components 20 or 120 are adjacent to the patient's skin. Preferably, local-anesthetic components 16 and 116 directly overlays the surgically closed wound 18 for effective administration of the local anesthetic directly to wound 18. The opioid components 20 and 120 are positioned for systemic delivery of the opioid through intact skin and, preferably, do not directly contact wound 18.

### 5. EXAMPLES

### 5.1 Example 1

### Preparation of a Patch of the Invention

An adhesive matrix comprising a mixture of lidocaine in acrylate polymer adhesive is prepared in the following manner. About 60 g acrylate polymer (Durotak® 387-2052) and about 50 g lidocaine are dissolved in ethyl acetate. The concentration of lidocaine is about 15 wt/wt percent wet weight.

The local-anesthetic component of the patch is then prepared by completely coating a 16 cm×10,000 cm×75 μm polyester film backing with the lidocaine acrylate matrix using a Warner Mathis thickness coater. The thickness of the wet film is about 270 μm.

The coated film is dried at a rate of about three feet per minute through a 9 foot temperature zone at a temperature gradient of about 60° C. to about 90° C. in a KTF oven to evaporate the ethyl acetate yielding the local-anesthetic component. The dry adhesive lidocaine matrix film has a thickness of about 160 μm and the lidocaine concentration is about 20 wt/wt percent dry weight.

The dry film is then laminated with a second sheet of 16 cm×10,000 cm×75 μm polyester film, thus giving the dry lidocaine matrix sandwiched between the two polyester films, referred to herein as the “lidocaine matrix laminate”. The lidocaine matrix laminate is run through a Mark Andy slitting and die cutting machine to give 4 rolls of 4 cm×10,000 cm lidocaine matrix laminates.

The opioid component of the patch is prepared in essentially the same manner. Fentanyl and acrylate polymer (Durotak® 387-20 52) are dissolved in ethyl acetate. The concentration of fentanyl is about 3.2 wt/wt percent wet weight. Then a 16 cm×10,000 cm×75 μm polyester film backing is completely coated with the opioid acrylate matrix using a Warner Mathis thickness coater. The thickness of the wet film is about 270 μm.

The coated film is dried at a rate of about three feet per minute through a 9 foot temperature zone at a temperature gradient of about 60° C. to about 90° C. in a KTF oven to evaporate the ethyl acetate to yield the opioid component. The dry adhesive opioid matrix film so formed has a thickness of about 160 μm and the concentration of fentanyl is about 5 wt/wt percent dry weight.

The dry opioid film is then laminated with a second sheet of 16 cm×10,000 cm×75 μm polyester film, thus giving the dry opioid matrix sandwiched between the two polyester films, referred to herein as the “opioid matrix laminate”. The opioid matrix laminate is run through a Mark Andy slitting and die cutting machine to give 16 rolls of 1 cm×10,000 cm opioid matrix laminates.

A 10,000 cm long roll having a 4 cm strip of lidocaine matrix laminate and a 1 cm wide opioid matrix laminate on either side is produced by running two rolls of 1 cm×10,000 cm opioid matrix laminate and one roll of 4 cm×10,000 cm lidocaine matrix laminate through a converter and winder. This active side of this roll is then unwound onto a permeable membrane and release liner respectively and then die cut into 15 cm long strips. These strips are sterilized and packaged using well-known methods.

### 5.2 Example 2

### Treatment of Incisional Pain Using a Patch of the Invention

A mesh surgical hernia repair is preformed in a surgical center on a patient suffering from a ventral or incisional hernia. A 5 to 6 inch incision is made in the patient's abdomen over the hernial defect. The abdominal hernia defect is repaired with a subfascial insertion of mesh (Kugel Type repair) with fixation and layered closure of the subcutaneous tissue and subcuticular. The surgical procedure has the pain components of incisional and deep peritoneal pain (peripheral and central pain receptor sites).

A sterile patch of the invention is applied to the wound in the operating room, directly after the surgery. Such application immediately after surgery provides preemptive anesthesia. Application is accomplished by pressing the patch firmly in place with the palm of the hand for thirty seconds, making sure the contact is complete, especially around the edges. The local-anesthetic component is contacted directly to the surgically closed wound. The surrounding opioid component is contacted with the intact skin surrounding the wound.

The Patient is monitored in the outpatient surgical office with reapplication of the pain patch at 72 hour intervals' for 1 week duration.

The present invention is not to be limited in scope by the specific embodiments disclosed in the examples, which are intended as illustrations of a few aspects of the invention. Any embodiments that are functionally equivalent are within the scope of this invention. Indeed, various modifications of the invention in addition to those shown and described herein will become apparent to those skilled in the art and are intended to fall within the scope of the appended claims. All cited references are hereby incorporated herein in their entireties by reference.

1. A patch comprising a local anesthetic and an opioid, wherein the opioid comprises an amount sufficient for transdermal delivery and systemic treatment of pain and the local anesthetic comprises an amount sufficient for intradermal delivery and local treatment of pain.

2. The patch of claim 1, wherein the opioid comprises fentanyl, buprenorphine, sufentanil, or hydromorphone or a pharmaceutically-acceptable salts thereof, or a mixture thereof.

3. The patch of claim 1, wherein the local anesthetic comprises lidocaine, tetracaine, bupivacaine, prilocalne, mepivacaine, procaine, chloroprocaine, ropivacaine, dibucaine, etidocaine, or benzocaine or a pharmaceutically acceptable salt thereof.

4. The patch of claim 1, wherein a local-anesthetic component comprises the local anesthetic and an opioid component comprises the opioid.

5. The patch of claim 4, wherein the opioid component surrounds the local anesthetic component.

6. The patch of claim 4, wherein the local-anesthetic component comprises of from about 0.1 mg/cm² to about 50 mg/cm² of the local anesthetic.

7. The patch of claim 4, wherein a dose of the opioid is an amount that provides pain relief equivalent to that provided by about 20 mg to about 80 mg of morphine orally administered over a 24 hour period.

8. The patch of claim 4, wherein the opioid release rate is of from about 0.5 μg/h to about 3,000 μg/h for every cm² of the opioid component.

9. The patch of claim 1, wherein the opioid comprises fentanyl.

10. The patch of claim 9, wherein the local anesthetic comprises lidocaine.

11. The patch of claim 9, wherein the patch delivers a dosage of the fentanyl of from about 10 μg/h to about 20 μg/h over a period of about 72 hours.

12. A method of treating, ameliorating or alleviating pain in a mammal comprising locally administering a therapeutically effective amount of a local anesthetic to the site of the pain and transdermally administering a therapeutically effective amount of an opioid to intact skin by way of one or more patches to a mammal in need of such treatment.

13. The method of claim 12, wherein the site of the pain comprises a wound.

14. The method of claim 13, wherein the wound resulted from a surgical procedure.

15. The method of claim 14, wherein the surgical procedure comprises laparoscopy, herniaplasty, breast biopsy, or excision of a subcutaneous tumor.

16. The method of claim 13, wherein the wound comprises a surgically closed wound.

17. The method of claim 12, wherein the opioid comprises fentanyl, buprenorphine, sufentanil, or hydromorphone or a pharmaceutically-acceptable salts thereof, or a mixture thereof.

18. The method of claim 12, wherein the local anesthetic comprises lidocaine, tetracaine, bupivacaine, prilocalne, mepivacaine, procaine, chloroprocaine, ropivacaine, dibucaine, etidocaine, or benzocaine or a pharmaceutically acceptable salt thereof.

19. The method of claim 12, wherein a local-anesthetic component comprises the local anesthetic and an opioid component comprises the opioid.

20. The method of claim 19, wherein the opioid component surrounds the local anesthetic component.

21. The method of claim 19, wherein the local-anesthetic component comprises of from about 0.1 mg/cm² to about 50 mg/cm² of the local anesthetic.

22. The method of claim 18, wherein a dose of the opioid is an amount that provides pain relief equivalent to that provided by about 20 mg to about 80 mg of morphine orally administered over a 24 hour period.

23. The method of claim 19, wherein the opioid release rate is of from about 0.5 μg/h to about 3,000 μg/h for every cm² of the opioid component.

24. The method of claim 12, wherein the opioid comprises fentanyl.

25. The method of claim 24, wherein the local anesthetic comprises lidocaine.

26. The method of claim 24, wherein the patch delivers a dosage of the fentanyl of from about 10 μg/h to about 20 μg/h over a period of about 72 hours.

27. A kit comprising a local anesthetic component and an opioid component, wherein the opioid component comprises an opioid for transdermal delivery and the local anesthetic component comprises a local anesthetic for local delivery, and wherein the amounts of the opioid and the local anesthetic are sufficient for treatment of pain caused by a surgically closed wound.

28. The kit of claim 27, wherein a first patch comprises the opioid component and a second patch comprises the local anesthetic component.

29. The kit of claim 27, wherein the opioid comprises fentanyl, buprenorphine, sufentanil, or hydromorphone or a pharmaceutically-acceptable salts thereof, or a mixture thereof.

30. The kit of claim 27, wherein the local anesthetic comprises lidocaine, tetracaine, bupivacaine, prilocalne, mepivacaine, procaine, chloroprocaine, ropivacaine, dibucaine, etidocaine, or benzocaine or a pharmaceutically acceptable salt thereof.

31. The kit of claim 27, wherein the local-anesthetic component comprises of from about 0.1 mg/cm² to about 50 mg/cm² of the local anesthetic.

32. The kit of claim 27, wherein a dose of the opioid is an amount that provides pain relief equivalent to that provided by about 20 mg to about 80 mg of morphine orally administered over a 24 hour period.

33. The kit of claim 27, wherein the opioid release rate is of from about 0.5 μg/h to about 3,000 μg/h for every cm² of the opioid component.

34. The kit of claim 27, wherein the opioid comprises fentanyl.

35. The kit of claim 34, wherein the local anesthetic comprises lidocaine.

36. The kit of claim 34, wherein the opioid component delivers a dosage of the fentanyl of from about 10 μg/h to about 20 μg/h over a period of about 72 hours.
